(19) United States
(12) Patent Application Publication
(10) Pub. No.: US 2022/0097057 A1
(43) Pub. Date: Mar. 31, 2022

(54) NUCLEIC ACID DETECTION HOST AND NUCLEIC ACID DETECTION DEVICE

(71) Applicant: iCare Diagnostics International Co. Ltd., New Taipei City (TW)

(72) Inventor: CHIH-NAN LIN, New Taipei (TW)

(21) Appl. No.: 17/488,602

(22) Filed: Sep. 29, 2021

Related U.S. Application Data
(60) Provisional application No. 63/085,368, filed on Sep. 30, 2020.

(30) Foreign Application Priority Data
May 31, 2021 (CN) 202110604892.6

(51) Int. Cl. B01L 3/00; G01N 27/447; B01L 7/00; C12Q 1/6806

(52) U.S. Cl. CPC B01L 3/502715; G01N 27/44743; G01N 27/44726; B01L 3/502723; B01L 2300/021; B01L 7/52; C12Q 1/6806; B01L 3/54; B01L 3/50273

(57) ABSTRACT

A nucleic acid detection host includes a host body, a detection kit installation area, a sample heating area, a sampling area, and an image collection unit. The detection kit installation area is configured to detachably install a nucleic acid detection kit. The sampling area is disposed above the detection kit installation area and is connected to the detection kit installation area. The sampling area is configured to add a detection solution into the nucleic acid detection kit. The image collection unit is configured to collect an image of the nucleic acid detection kit. A nucleic acid detection device including the nucleic acid detection host is also disclosed. The nucleic acid detection device has a simple structure, which is portable, flexible, and convenient, and can be used at home.

### FIELD

The subject matter relates to nucleic acid detection, and more particularly, to a nucleic acid detection host and a nucleic acid detection device with the nucleic acid detection host.

### BACKGROUND

Molecular diagnosis, morphological detection, and immunological detection are mostly carried out in laboratories. Detection processes are time-consuming, inefficient, and inflexible, and detection devices are generally not portable. Therefore, detection cannot be carried out anytime and anywhere. Especially, patients with highly infectious virus may infect others on the way to the laboratories, which has potential safety hazards.

### BRIEF DESCRIPTION OF THE DRAWINGS

Implementations of the present technology will now be described, by way of example only, with reference to the attached figures.

FIG. 1 is a diagrammatic view of an embodiment of a nucleic acid detection host according to the present disclosure.

FIG. 2 is a cross-sectional view taken along II-II of FIG. 1.

FIG. 3 is a diagrammatic view of internal structures of the nucleic acid detection host in FIG. 1.

FIG. 4 is a diagrammatic view of an embodiment of a clamping structure according to the present disclosure.

FIG. 5 is a diagrammatic view of an embodiment of an image collection unit and a nucleic acid detection kit according to the present disclosure.

FIG. 6 is a diagrammatic view of an embodiment of a nucleic acid detection device according to the present disclosure.

FIG. 7 is a diagrammatic view of an embodiment of a nucleic acid detection kit according to the present disclosure.

FIG. 8 is an explosion diagrammatic view of the nucleic acid detection kit according to the present disclosure.

FIG. 9 is a diagrammatic view of an embodiment of a liquid transfer structure according to the present disclosure.

FIG. 10 is a diagrammatic view of an embodiment of a collection cup according to the present disclosure.

FIG. 11 is a diagrammatic view of an embodiment of a reagent package according to the present disclosure.

FIG. 12 to FIG. 16 are diagrammatic views showing steps of a nucleic acid detecting process according to the present disclosure.

FIG. 17 is a flowchart of a method for detecting nucleic acid according to the present disclosure.

### DETAILED DESCRIPTION

It will be appreciated that for simplicity and clarity of illustration, where appropriate, reference numerals have been repeated among the different figures to indicate corresponding or analogous components. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale, and the proportions of certain parts may be exaggerated to better illustrate details and features of the present disclosure.

The term “comprising,” when utilized, means “including, but not necessarily limited to”; it specifically indicates open-ended inclusion or membership in the so-described combination, group, series, and the like.

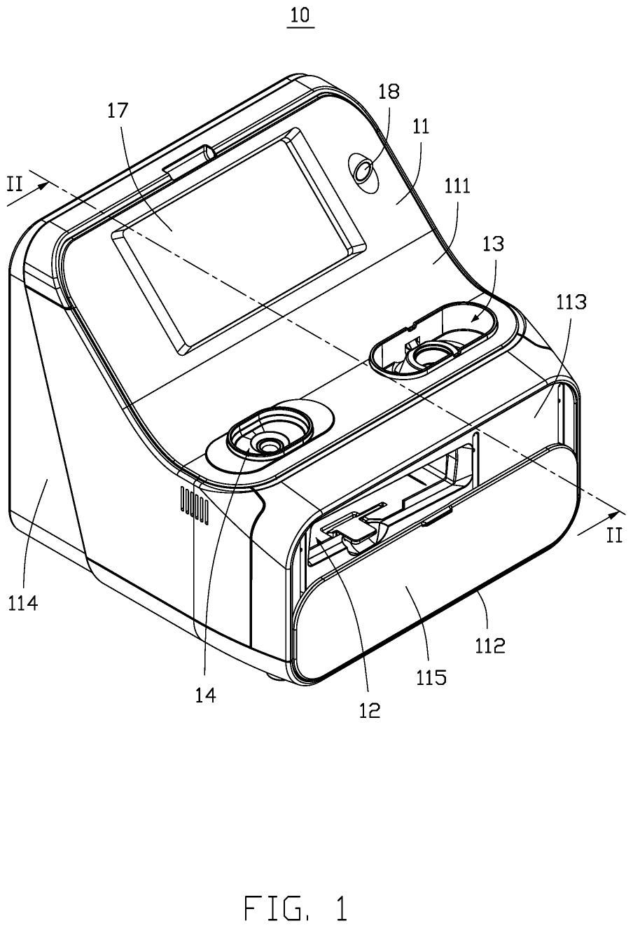

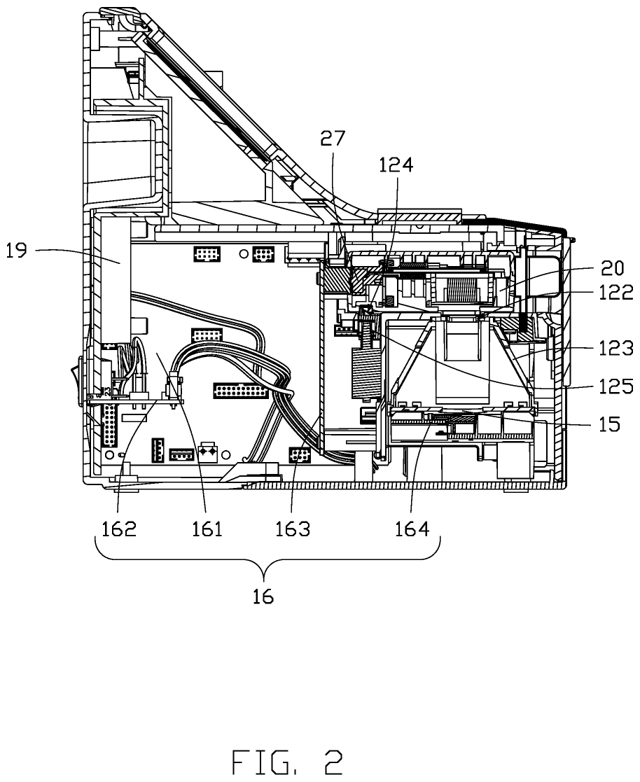

FIGS. 1 and 2 illustrate a nucleic acid detection host 10, which includes a host body 11, a detection kit installation area 12, a sample heating area 13, a sampling area 14, an image collection unit 15, and a controller 16. The detection kit installation area 12, the sample heating area 13, the sampling area 14, the image collection unit 15, and the controller 16 are disposed on the host body 11. The sample heating area 13 and the image collection unit 15 are electrically connected to the controller 16.

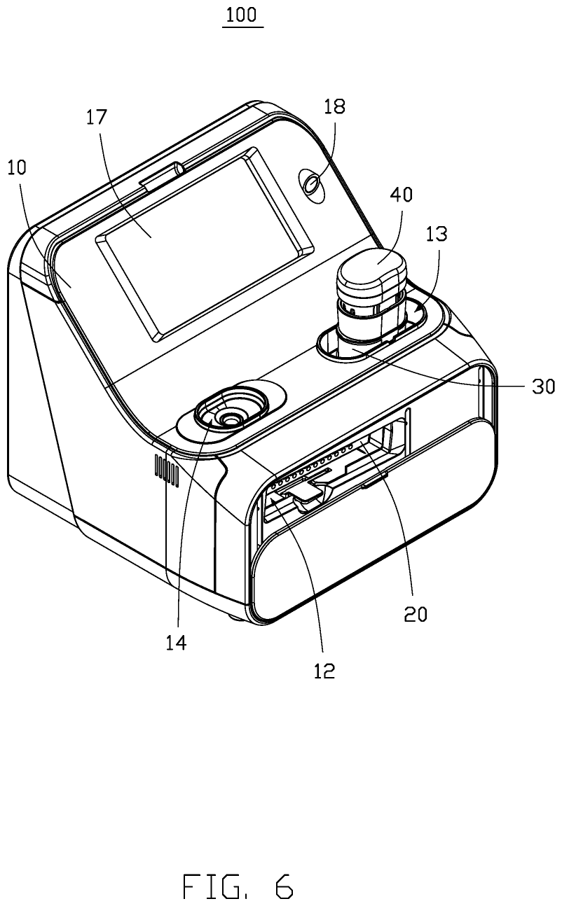

The sample heating area 13 is used to collect a nucleic acid sample of an object. The nucleic acid sample is mixed with a detection agent (such as a buffer solution) in the sample heating area 13 to form a detection solution. The sample heating area 13 is further used to heat the detection solution under the control of the controller 16. The sampling area 14 is disposed above and connected to the detection kit installation area 12. Referring to FIG. 6, a nucleic acid detection kit 20 is detachably installed in the detection kit installation area 12. The nucleic acid detection kit 20 is electrically connected to the controller 16. The sampling area 14 is used to add the detection solution into the nucleic acid detection kit 20. Then, the detection solution undergoes a PCR amplification reaction to form an amplification product, which further undergoes an electrophoretic detection in the nucleic acid detection kit 20. The image collection unit 15 is disposed on a side of the detection kit installation area 12 away from the sampling area 14. The image collection unit 15 is used to collect an image of the nucleic acid detection kit 20 in the detection kit installation area 12 under the control of the controller 16. The image is a fluorescent image of the electrophoretic detection, and a detection result can be obtained according to the fluorescent image.

Referring to FIG. 1, the host body 11 includes a first surface 111, a second surface 112 opposite the first surface 111, a first sidewall 113 connecting the first surface 111 and the second surface 112, and a second sidewall 114 opposite the first sidewall 113. The first sidewall 113 defines an opening of the detection kit installation area 12, and the nucleic acid detection kit 20 can be placed in the detection kit installation area 12 through the opening. The first surface 111 defines an opening of the sampling area 14 and an opening of the sample heating area 13.

Referring to FIGS. 1-3, the detection kit installation area 12 includes a mounting groove 121, an imaging port 122 disposed on a bottom surface of the mounting groove 121, a fixing box 123 disposed on a side of the imaging port 122 closed to the second surface 112, a detection kit clamping port 124 disposed on the bottom surface of the mounting groove 121, a clamping structure 125 disposed on a side of the detection kit clamping port 124 closed to the second surface 112, and a first sensor 126 disposed on the host body 11 outside the mounting groove 121. The first sensor 126 is connected to the controller 16. The nucleic acid detection kit 20 may be manually inserted into the mounting groove 121. The clamping structure 125 passes through the detection kit clamping port 124, and detachably clamps the bottom of the nucleic acid detection kit 20 in the mounting groove 121. The sampling area 14 is connected to the mounting groove 121 through a through hole (not shown in the figures), so that samples can be added into the nucleic acid detection kit 20. The image collection unit 15 is disposed in the fixing box 123 to collect the fluorescent image in the nucleic acid detection kit 20 through the imaging port 122. The first sensor 126 senses whether the nucleic acid detection kit 20 is inserted into the mounting groove 121, and transmits a signal to the controller 16 to control the nucleic acid detection kit 20 to start the nucleic acid detection.

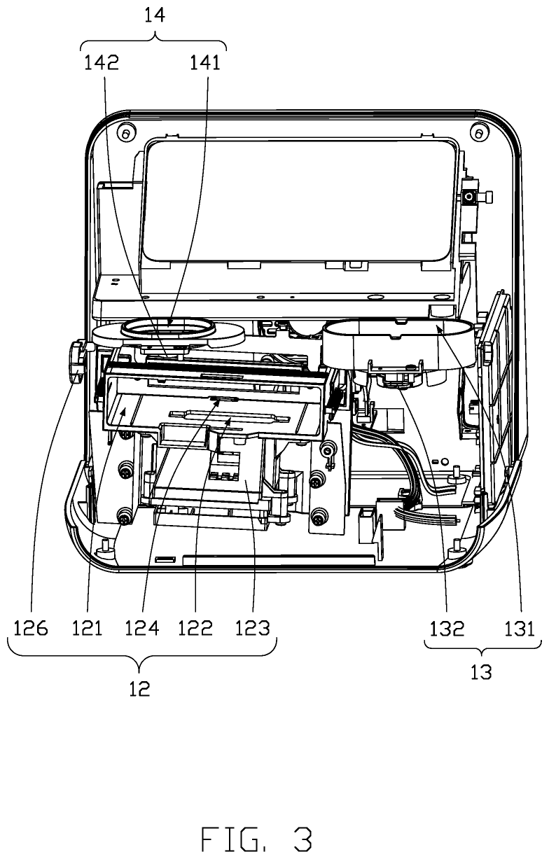

Referring to FIGS. 2 and 3, the mounting groove 121 is inclined relative to the first surface 111, which can make the nucleic acid detection kit 20 be placed obliquely in the mounting groove 121. In an embodiment, relative to the first surface 111, a height of an end of the mounting groove 121 closed to the sampling area 14 is higher than a height of another end of the mounting groove 121 away from the sampling area 14. Bubbles will be generated during a PCR amplification reaction in the nucleic acid detection kit 20. The bubbles may stay in and block a flow path of microbeads in the nucleic acid detection kit 20, so that the microbeads cannot move in the flow path that causes a failure of the nucleic acid detection. Therefore, the mounting groove 121 is designed to be inclined, so that the nucleic acid detection kit 20 can be placed obliquely, and the bubbles generated by the PCR amplification reaction can be discharged out without hindering the movement of the microbeads.

A shape of the mounting groove 121 may be designed according to a shape of the nucleic acid detection kit 20. In an embodiment, the mounting groove 121 is substantially rectangular.

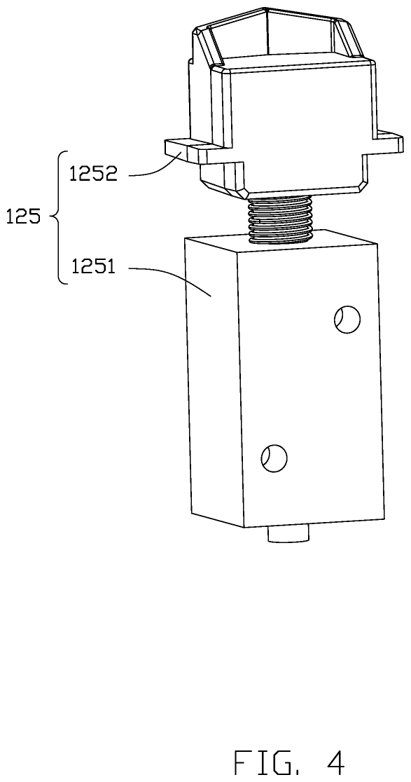

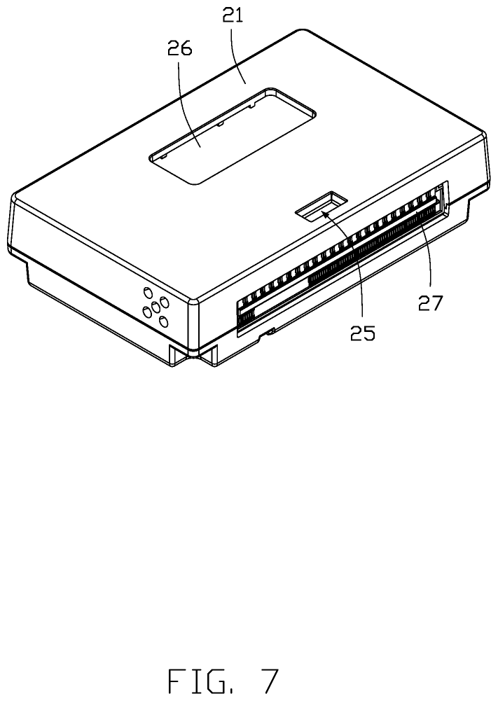

Referring to FIGS. 2, 4, and 7, the clamping structure 125 includes a solenoid valve 1251 and a top block 1252 disposed on the solenoid valve 1251. A clamping groove 25 matching the top block 1252 is disposed on a bottom surface of the nucleic acid detection kit 20. The top block 1252 corresponds to the detection kit clamping port 124. The solenoid valve 1251 is connected to the controller 16. When the first sensor 126 senses that the nucleic acid detection kit 20 is inserted into the mounting groove 121, the solenoid valve 1251, when energized, can be lifted up and push the top block 1252 into the clamping groove 25 to fix the nucleic acid detection kit 20. After the nucleic acid detection, the solenoid valve 1251 is energized to eject the nucleic acid detection kit 20 from the mounting groove 121. By setting the solenoid valve 1251 and the first sensor 126, automatic locking and automatic ejection of the nucleic acid detection kit 20 can be realized.

Referring to FIG. 3, the first sensor 126 is used to sense whether the nucleic acid detection kit 20 is inserted into or ejected from the mounting groove 121, and the sensing result is used to initiate or end the nucleic acid detection. When the first sensor 126 detects that the nucleic acid detection kit 20 is inserted into the mounting groove 121, the nucleic acid detection is started. When the first sensor 126 detects that the nucleic acid detection kit 20 is ejected from the mounting groove 121, the nucleic acid detection is ended.

In an embodiment, referring to FIG. 3, the imaging port 122 is substantially rectangular. A size of the imaging port 122 makes sure that the image collection unit 15 can collect a complete fluorescent image in the nucleic acid detection kit 20 through the imaging port 122.

Referring to FIGS. 1, 3, and 5, the fixing box 123 has an inverted conical funnel structure. A sidewall of the fixing box 123 is inclined with respect to the first surface 111. A size of an end of the fixing box 123 close to the first surface 111 is smaller than a size of another end of the fixing box 123 away from the first surface 111. The end of the fixing box 123 close to the first surface 111 is connected to the mounting groove 121 through the imaging port 122. The image collection unit 15 corresponds to the imaging port 122. The inclined sidewall of the fixing box 123 can focus and concentrate light passing through the imaging port 122 during imaging.

In an embodiment, the inner surface of the sidewall of the fixing box 123 is covered by a reflective coating, which can reflect the light into the imaging port 122.

In an embodiment, referring to FIGS. 1 and 3, an opening of the mounting groove 121 is on the first sidewall 113. A front baffle 115 is disposed at the opening of the mounting groove 121, which is used to close or open the opening. The front baffle 115 is slidably disposed at the opening of the mounting groove 121. The front baffle 115 can move from the first surface 111 to the second surface 112 along the first sidewall 113 to open the opening of the mounting groove 121. The front baffle 115 can also move from the second surface 112 to the first surface 111 along the first sidewall 113 to close the opening of the mounting groove 121.

Referring to FIGS. 2 and 3, the sample heating area 13 includes a holding tank 131 and a heating block 132 disposed at the bottom of the holding tank 131. The heating block 132 is electrically connected to the controller 16. The controller 16 can energize the heating block 132 to heat the heating block 132. The controller 16 can also detect a temperature and a heating time of the heating block 132 through a temperature sensor (not shown in the figures) and a time relay (not shown in the figures), respectively. In an embodiment, the heating temperature of the heating block 132 is about 95°, and the heating time is about 5 minutes. After heating, the heating block 132 is cooled at room temperature or at a specific temperature (such as below 40°).

In an embodiment, the heating block 132 is an aluminum block, a copper block, or other heating structures such as heating wire, heating coating, and heating sheet. At the same time, the sample heating area 13 is also provided with a second sensor (not shown in the figures). Referring to FIG. 6, whether a collection cup 30 is inserted into the holding tank 131 can be sensed by the second sensor. When the collection cup 30 is inserted into the holding tank 131, the second sensor sends a trigger signal to the controller 16 to initiate the heating process.

In an embodiment, an opening of the holding tank 131 is disposed on the first surface 111, and the opening of the holding tank 131 is substantially elliptical. A shape of the holding tank 131 can be specifically designed according to a shape of the collection cup 30.

Referring to FIG. 3, the sampling area 14 includes a sampling groove 141 and a sampling channel 142. An opening of the sampling groove 141 is disposed on the first surface 111. The sampling channel 142 is inserted into the mounting groove 121. An end of the sampling channel 142 away from the sampling groove 141 is connected to the nucleic acid detection kit 20. The detection solution enters the nucleic acid detection kit 20 in the mounting groove 121 through the sampling channel 142.

In an embodiment, the sampling groove 141 is funnel-shaped.

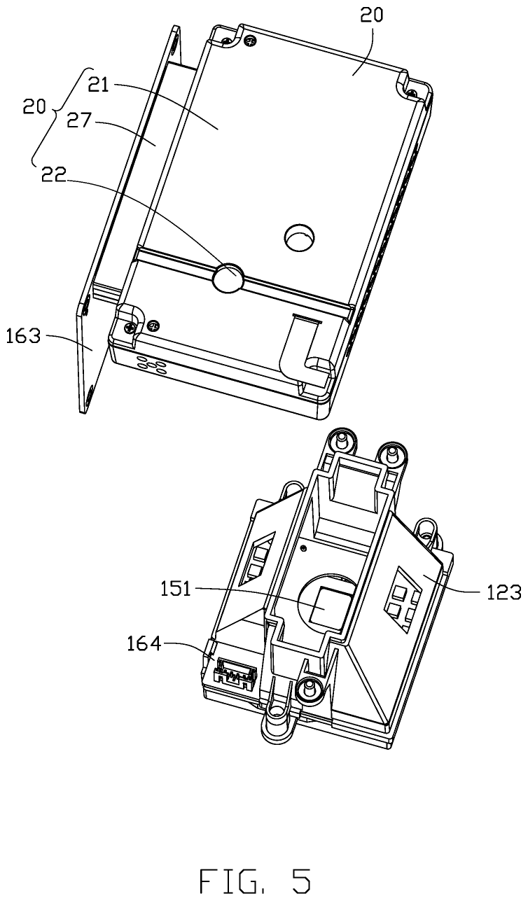

Referring to FIGS. 2 and 5, the image collection unit 15 includes a light source (not shown) and an image collector 151 each electrically connected to the controller 16. The light source is used to emit light to the imaging port 122 under the control of the controller 16. The image collector 151 is used to collect the light to form fluorescent images in the nucleic acid detection kit 20 under the control of the controller 16.

Referring to FIGS. 2, 3, and 5, the controller 16 includes a main control board 161, a power supply board 162, a detection kit control board 163, and an image acquisition control board 164. The power supply board 162 is electrically connected to the main control board 161, the detection kit control board 163, and the image acquisition control board 164 to supply power thereto. The detection kit control board 163 is electrically connected to the nucleic acid detection kit 20 to control the nucleic acid detection process. The image acquisition control board 164 is electrically connected to the image collection unit 15 to control the light source to emit the light and also control the image collector 151 to collect the fluorescent image of the nucleic acid detection kit 20. The heating block 132, the first sensor 126, and the second sensor are electrically connected to the main control board 161.

In an embodiment, the image acquisition control board 164 includes an image processor (not shown in the figures). The fluorescent image collected by the image collector 151 is transmitted to the image processor for processing, and a processed image is further output.

In an embodiment, the controller 16 further includes a memory (not shown) for storing detection results and information related to the detection process.

Referring to FIGS. 1 and 2, the nucleic acid detection host 10 further includes a display screen 17 and a camera 18, which are electrically connected to the main control board 161. The display screen 17 is used to display an operation interface to allow a user to set operation parameters, and also used to display the fluorescent images. The camera 18 is used to record an operation process of the user, and to collect relevant information of the detection solution (such as information indicating a source of the nucleic acid sample).

Referring to FIG. 2, the nucleic acid detection host 10 further includes a heat dissipation structure 19, which is electrically connected to the main control board 161 to dissipate heat for the nucleic acid detection host 10.

In an embodiment, the heat dissipation structure 19 is a fan. The heat dissipation structure 19 is disposed on the second sidewall 114. A plurality of heat dissipation vents is disposed on the host body 11 to discharge the heat inside the nucleic acid detection host 10.

Cooperation between the nucleic acid detection host 10 and the nucleic acid detection kit 20 can perform the PCR amplification reaction and the electrophoresis detection. The fluorescent image displayed on the display screen 17 is the image of the electrophoresis detection. The nucleic acid detection host 10 integrates the heating, sampling, detecting, and result outputting into one equipment. Thus, the nucleic acid detection host 10 has a simple structure, which is portable, flexible, and convenient, and can be used at home.

FIG. 6 illustrates a nucleic acid detection device 100 according to the present disclosure. The nucleic acid detection device 100 includes the nucleic acid detection host 10, the nucleic acid detection kit 20, the collection cup 30, and a liquid transfer structure 40. The nucleic acid detection kit 20 is detachably disposed in the mounting groove 121. The collection cup 30 is detachably disposed in the holding tank 131. The liquid transfer structure 40 is detachably connected to the collection cup 30 and detachably disposed in the sampling groove 141. The collection cup 30 is used to receive the detection solution. The liquid transfer structure 40 is used to quantitatively absorb the detection solution from the collection cup 30 and add the detection solution into the nucleic acid detection kit 20 through the sampling groove 141. The nucleic acid detection kit 20 is used to perform the PCR amplification reaction and the electrophoresis detection successively.

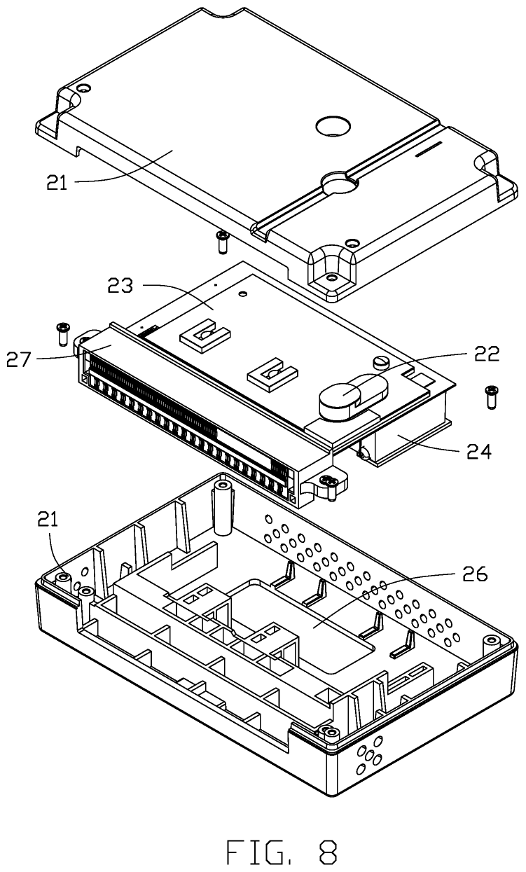

Referring to FIGS. 2, 5, 7, and 8, the nucleic acid detection kit 20 includes a kit body 21, a sampling port 22 disposed on the kit body 21, a detection chip 23, an electrophoresis box 24, detecting window 26, and a connector 27. The sampling port 22 is disposed close to the sampling groove 141. An end of the sampling channel 142 away from the sampling groove 141 is connected to the sampling port 22. The sampling port 22 is used to add the detection solution into the detection chip 23. The detection chip 23 and the electrophoresis box 24 connected together are disposed in the kit body 21. The detection chip 23 is used to perform the PCR amplification reaction. The electrophoresis box 24 is used to perform the electrophoresis detection. The detecting window 26 is disposed on a surface of the kit body 21 close to the imaging port 122. The detecting window 26 is disposed to correspond to the electrophoresis box 24. The image collection unit 15 acquires the fluorescent images of the electrophoresis box 24 through the imaging port 122 and the detecting window 26. The clamping groove 25 is disposed on a side of the kit body 21 close to the image collection unit 15. The connector 27 is disposed on one side of the kit body 21 close to the detection kit control board 163. The connector 27 is electrically connected to the detection kit control board 163, the detection chip 23, and the electrophoresis box 24. The electrophoresis box 24 and the detection chip 23 are disposed in the kit body 21. After the PCR amplification reaction is completed, the electrophoresis detection can be carried out automatically. The two processes are performed in a single equipment, and the sampling accuracy is controlled accurately. The nucleic acid detection kit 20 integrates with the detection chip 23 and the electrophoresis box 24, which has a small size, and is suitable for the nucleic acid detection device 100.

In an embodiment, the nucleic acid detection kit 20 is disposable. The nucleic acid detection kit 20 has no need to be cleaned after used.

In an embodiment, the nucleic acid detection kit 20 has substantially a cubic structure.

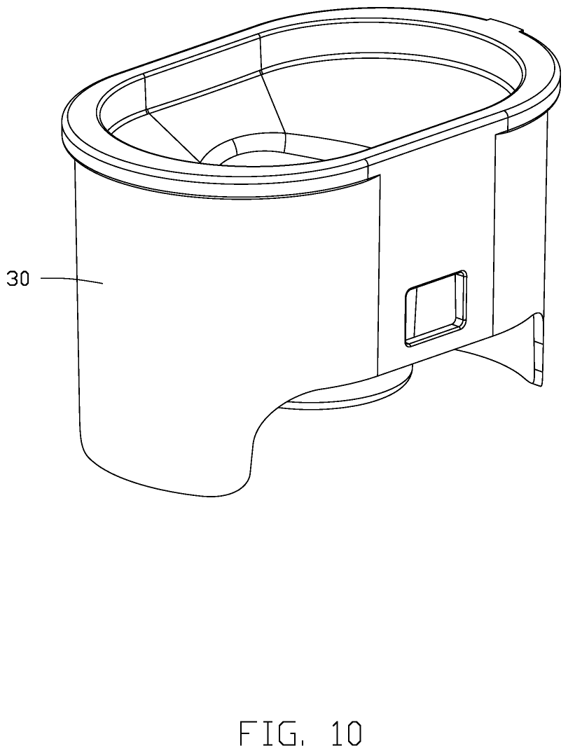

Referring to FIGS. 6 and 10, the collection cup 30 and the liquid transfer structure 40 can be clamped together. The collection cup 30 is used to collect the nucleic acid sample (such as saliva or other liquid sample), which is mixed with a detection reagent to form the detection solution. The detection solution is then heated in the sample heating area 13. The liquid transfer structure 40 is used to quantitatively absorb the detection solution from the collection cup 30, and add the detection solution into the nucleic acid detection kit 20 through the sampling groove 141.

In an embodiment, a conical groove is disposed inside the collection cup 30. After spitting saliva into the collection cup 30, the saliva can be concentrated in the bottom of the conical groove to facilitate the collection of a small amount of nucleic acid sample.

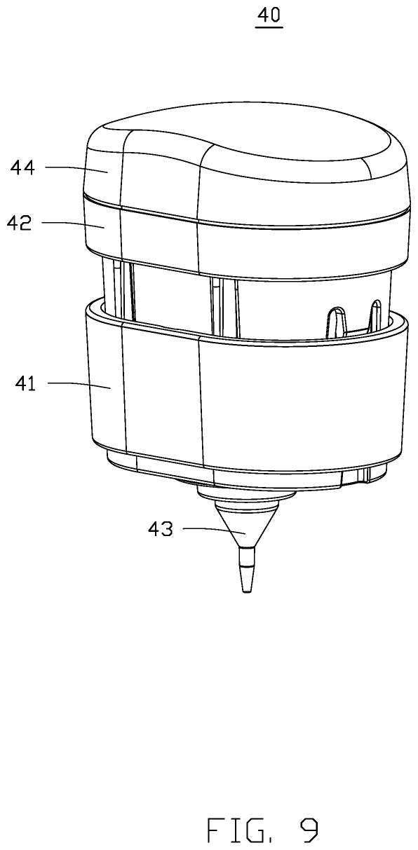

Referring to FIG. 9, the liquid transfer structure 40 includes a lower shell 41, an upper shell 42, a liquid extraction assembly 43, and a pressing key 44. The upper shell 42 is clamped with the lower shell 41. The liquid extraction assembly 43 is disposed in the lower shell 41 and the upper shell 42, and an end of the liquid extraction assembly 43 is extended out of the lower shell 41. The pressing key 44 is disposed on the top of the upper shell 42. In an embodiment, the liquid extraction assembly 43 includes an elastic liquid extraction structure. When in use, the upper shell 42 can be pressed down when the pressing key 44 is pressed, so that the upper shell 42 moves downward along a sidewall of the lower shell 41 to compress the liquid extraction assembly 43. Thus, air inside the elastic liquid extraction structure is extracted out, causing the elastic liquid extraction structure to absorb the detection solution. After absorbing the detection solution, the liquid extraction assembly 43 pushes the upper shell 42 to return to its original position automatically. When the detection solution in the liquid extraction assembly 43 needs to be discharged out, the pressing key 44 can be pressed again to move the upper shell 42 downward relative to the lower shell 41, which further squeezes the liquid extraction assembly 43 to discharge the detection solution out. The compression degree of the elastic liquid extraction structure can be controlled to control the volume of the detection solution, so as to achieve quantitative liquid extraction. The liquid transfer structure 40 has the advantages of simple overall structure, low cost, convenient operation, and can achieve the purpose of quantitative

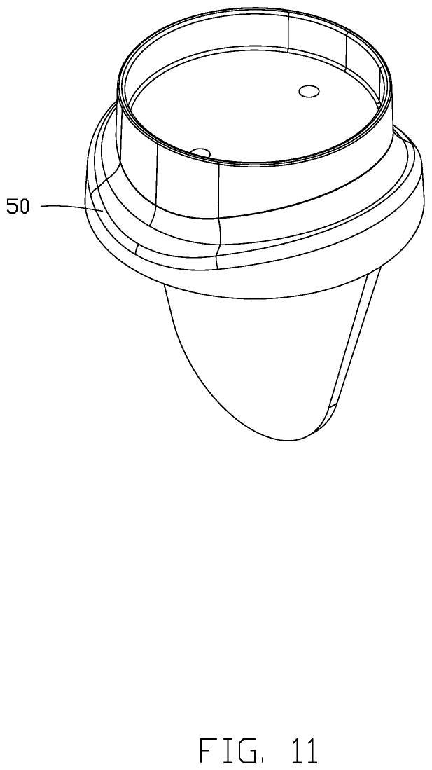

Referring to FIGS. 6 and 11, the nucleic acid detection device 100 further includes a reagent package 50 for storing a detection reagent (such as a buffer solution). The detection reagent is quantitatively placed in the reagent package 50. The reagent package 50 added into the collection cup 30 can be mixed with the nucleic acid sample to form the detection solution.

In an embodiment, the reagent package 50 is a groove structure with a handle. A detection reagent required for the nucleic acid detection is placed in the groove structure, and an opening of the reagent package 50 is sealed by a sealing film. When in use, the user can tear off the sealing film, grasp the handle, pour the detection reagent into the collection cup 30 containing the nucleic acid sample, and then put the collection cup 30 into the holding tank 131 for heating.

Before the nucleic acid detection, the nucleic acid detection kit 20, the collection cup 30, the liquid transfer structure 40, and the reagent package 50 are packed in a box. The nucleic acid detection kit 20, the collection cup 30, the liquid transfer structure 40, and the reagent package 50 can be provided with an identification code (such as a quick response code and a QR code) to avoid confusion. The identification code can only be set on the collection cup 30 to avoid confusion of the detection solution to be detected.

In an embodiment, the camera 18 is used to record the operation process of the user, and collect the identification code on the collection cup 30.

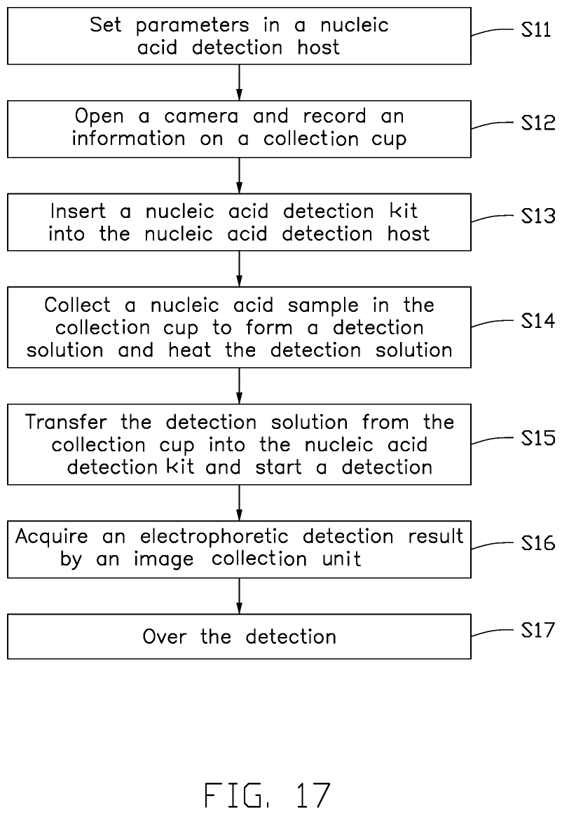

FIG. 17 illustrates a flowchart of a method for detecting the nucleic acid through the nucleic acid detection device 100 according to an embodiment. The method for detecting the nucleic acid through the nucleic acid detection device 100 is provided by way of example, as there are a variety of ways to carry out the method. The method can begin at block 11.

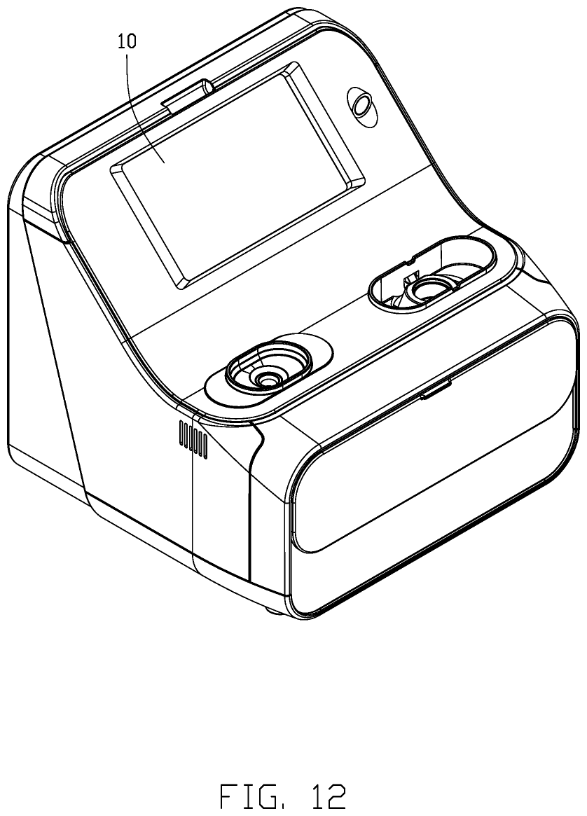

Block 11, referring to FIG. 12, operation parameters are set in the nucleic acid detection device 100. The nucleic acid detection host 10 is turned on and the operation parameters are set in the nucleic acid detection host 10 through the display screen 17.

In an embodiment, the operation parameters include the heating temperature and the heating time of the sample heating area 13, process parameters of the PCR amplification reaction, and process parameters of the electrophoresis detection.

Block 12, referring to FIG. 12, the information on the collection cup 30 is collected, and the operation process of the user is recorded.

The camera 18 is turned on to record the operation process of the user. The packaging box containing the nucleic acid detection kit 20, the collection cup 30, and the reagent package 50 is opened. Then the identification code on the collection cup 30 is collected by the camera 18 to collect relevant information of the nucleic acid sample. The collected information and video data can be uploaded and sent to a client for relevant personnel to view.

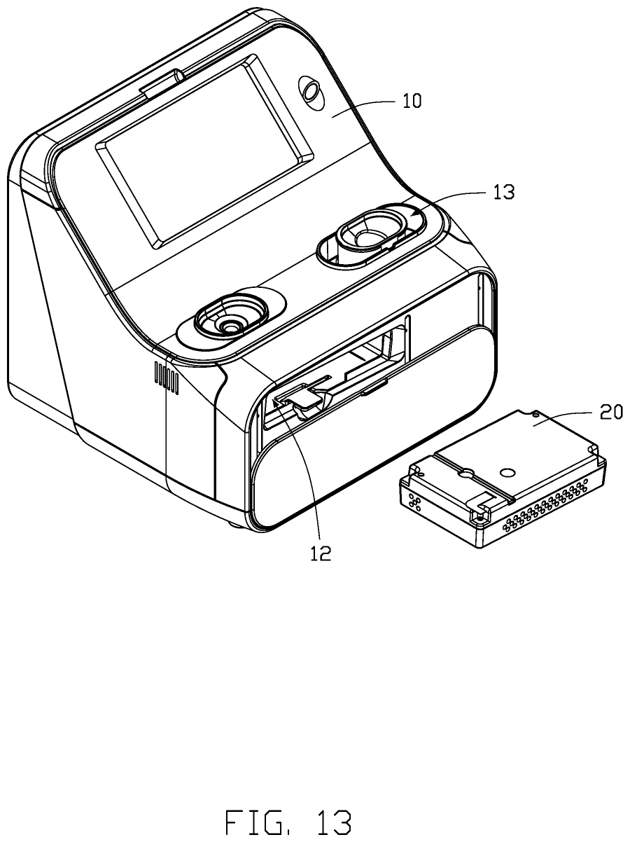

Block 13, referring to FIG. 13, the nucleic acid detection kit 20 is inserted into the mounting groove 121. The first sensor 126 senses the insertion of the nucleic acid detection kit 20, and then automatically initiates the nucleic acid detection.

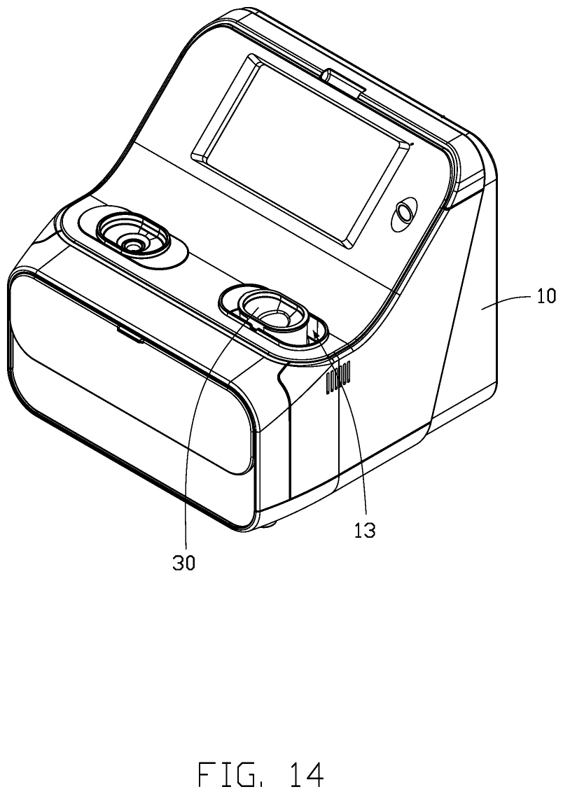

Block 14, referring to FIG. 14, the nucleic acid sample is collected by the collection cup 30 and mixed with a detection reagent to form a detection solution. The detection solution is then heated in the sample heating area 13.

In an embodiment, the nucleic acid sample (such as saliva) is collected by the collection cup 30. Then the detection reagent in the reagent package 50 is poured into the collection cup 30. The reagent package 50 is buckled at the opening of the collection cup 30. The collection cup 30 is covered and shaken up and down for 3-5 times to obtain the detection solution. Generally, the nucleic acid sample (the saliva) and the detection reagent can be mixed evenly by shaking the collection cup 30 up and down for 5 times. The collection cup 30 with the detection solution is insert into the holding tank 131. When the collection cup 30 is inserted into the holding tank 131, the second sensor sends a trigger signal to the controller 16 to initiate the heating process. The heating temperature is in a range from 90° to 100°, and the heating time is in a range from 3 to 8 min. Then the holding tank 131 is cooled to room temperature or below a preset temperature (such as below 40°). In an embodiment, the temperature sensor and the time relay are used to sense the heating temperature and the heating time.

In yet another embodiment, the saliva is collected by the collection cup 30 and then be heated in the holding tank 131. The heating temperature is in a range from 90° to 100°, and the heating time is in a range from 3 to 8 min. After heating, the saliva is cooled to room temperature or below a preset temperature (such as below 40°). After cooling, the detection reagent in the reagent package 50 is added into the collection cup 30 to mix with the saliva to form the detection solution.

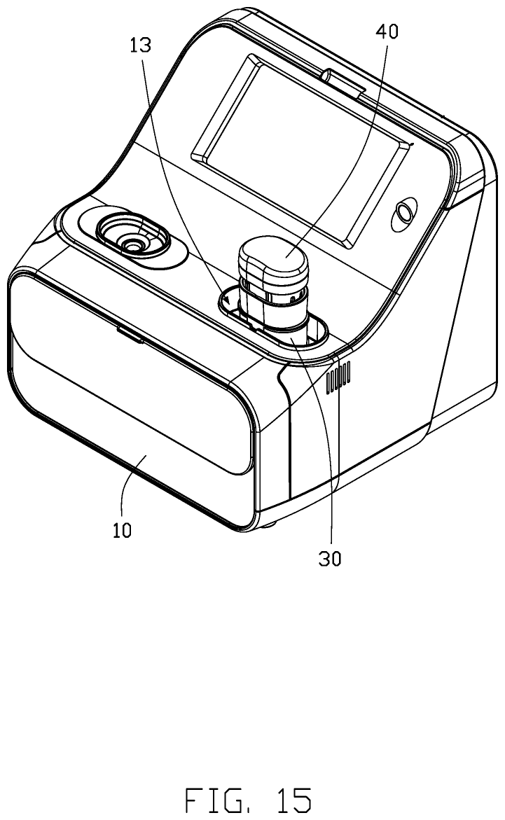

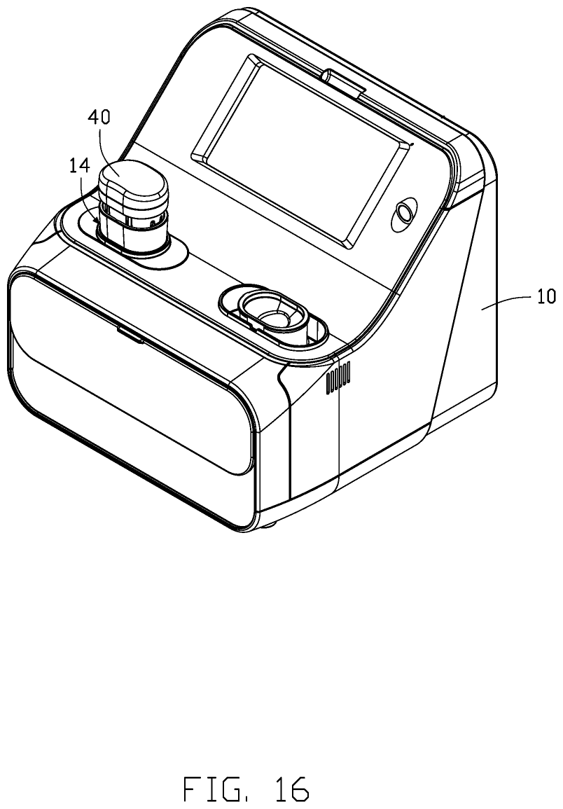

Block 15, referring to FIGS. 15 and 16, the detection solution is transferred from the collection cup 30 into the nucleic acid detection kit 20 for performing the PCR amplification reaction and the electrophoresis detection.

In an embodiment, the detection solution is quantitatively sucked 10-30 μl (preferably 20 μl) by the liquid transfer structure 40 from the collection cup 30 and is added into the nucleic acid detection kit 20. The detection solution containing the nucleic acid sample undergoes the PCR amplification reaction in the detection chip 23. After amplification, the detection solution is combined with a fluorescent reagent placed in the detection chip 23 to form a product with fluorescent groups. Then the product with fluorescent groups enters the electrophoresis box 24 from the detection chip 23 to undergo the electrophoresis detection.

Block 16, an electrophoretic detection result (such as the fluorescent image) is acquired by the image collection unit 15.

After the electrophoretic detection, the fluorescent image is acquired by the image collection unit 15 through the detecting window 26. The fluorescent image is processed by the image processor, and then displayed on the display screen 17. The fluorescent image can also be uploaded and sent to the client for the user to consult.

Block 17, the nucleic acid detection is over.

After the nucleic acid detection, the collection cup 30, the liquid transfer structure 40, and the nucleic acid detection kit 20 are removed from the nucleic acid detection device 100 and put into the packaging box for recycling.

The nucleic acid detection device 100 provided by the present disclosure can integrate the PCR amplification reaction and the electrophoresis detection of nucleic acid into in a single equipment through the cooperation of the nucleic acid detection host 10 and the nucleic acid detection kit 20. Thus, the nucleic acid detection device 100 has a simple structure, which is portable, flexible, and convenient, and can be used at home. At the same time, the detecting process is flexible, which does not need to be carried out in a professional laboratory.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure, up to and including, the full extent established by the broad general meaning of the terms used in the claims.

What is claimed is:

1. A nucleic acid detection host, comprising:
a host body;
a detection kit installation area;
a sample heating area;
a sampling area; and
an image collection unit;
wherein the detection kit installation area, the sample heating area, the sampling area, and the image collection unit are disposed on the host body, the detection kit installation area is configured to detachably install a nucleic acid detection kit therein, the sample heating area is configured to collect a detection solution and heat the detection solution, the sampling area is disposed above the detection kit installation area and connected to the detection kit installation area, the sampling area is configured to add the detection solution into the nucleic acid detection kit in the detection kit installation area, the image collection unit is disposed on a side of the detection kit installation area away from the sampling area, and is configured to collect an image of the nucleic acid detection kit.

2. The nucleic acid detection host of claim 1, wherein the detection kit installation area comprises a mounting groove, an imaging port disposed on a surface of the mounting groove away form the sampling area, a fixing box disposed on a side of the imaging port away from the sampling area, a detection kit clamping port disposed beside the imaging port, and a clamping structure corresponding to the detection kit clamping port,
the clamping structure passes through the detection kit clamping port to clamp the nucleic acid detection kit, the image collection unit is disposed in the fixing box, and is configured to collect the image through the imaging port.

3. The nucleic acid detection host of claim 2, wherein a height of an end of the mounting groove closed to the sampling area is higher than a height of another end of the mounting groove away from the sampling area.

4. The nucleic acid detection host of claim 2, wherein the clamping structure comprises a solenoid valve and a top block disposed on the solenoid valve, the top block corresponds to the detection kit clamping port, the solenoid valve is configured to push the top block into the mounting groove through the detection kit clamping port when energized, causing the top block to fix or loosen the nucleic acid detection kit.

5. The nucleic acid detection host of claim 1, wherein the sample heating area comprises a holding tank and a heating block disposed at a bottom of the holding tank.

6. The nucleic acid detection host of claim 1, further comprising a first sensor disposed on the detection kit installation area and a second sensor disposed on the sample heating area, wherein the first sensor is configured to sense whether the nucleic acid detection kit is inserted into the detection kit installation area, and the second sensor is configured to sense whether the detection solution is added into the sample heating area.

7. The nucleic acid detection host of claim 1, further comprising a display screen and a camera, wherein the display screen is configured to display the image of the nucleic acid detection kit, and the camera is configured to record identification information of the detection solution.

8. A nucleic acid detection device, comprising:
a nucleic acid detection host, comprising:
a host body;
a detection kit installation area;
a sample heating area;
a sampling area; and
an image collection unit,
wherein the detection kit installation area, the sample heating area, the sampling area, and the image collection unit are disposed on the host body, the detection kit installation area is configured to detachably install a nucleic acid detection kit therein, the sample heating area is configured to collect a detection solution and heat the detection solution, the sampling area is disposed above the detection kit installation area and connected to the detection kit installation area, the sampling area is configured to add the detection solution into the nucleic acid detection kit in the detection kit installation area, the image collection unit is configured on a side of the detection kit installation area away from the sampling area, and is configured to collect an image of the nucleic acid detection kit;
a collection cup;
a liquid transfer structure; and
the nucleic acid detection kit,
wherein the collection cup is detachably disposed in the sample heating area, the collection cup is configured to receive the detection solution, the liquid transfer structure is detachably disposed in the sampling area or the collection cup, the liquid transfer structure is configured to transfer the detection solution from the collection cup into the nucleic acid detection kit, the nucleic acid detection kit is configured to perform a PCR amplification reaction and an electrophoresis detection.

9. The nucleic acid detection device of claim 8, wherein the detection kit installation area comprises a mounting groove, an imaging port disposed on a surface of the mounting groove away form the sampling area, a fixing box disposed on a side of the imaging port away from the sampling area, a detection kit clamping port disposed beside the imaging port, and a clamping structure corresponding to the detection kit clamping port,
the clamping structure passes through the detection kit clamping port to clamp the nucleic acid detection kit, the image collection unit is disposed in the fixing box, and is configured to collect the image through the imaging port.

10. The nucleic acid detection device of claim 8, wherein a height of an end of the mounting groove closed to the sampling area is higher than a height of another end of the mounting groove away from the sampling area.

11. The nucleic acid detection device of claim 9, wherein the clamping structure comprises a solenoid valve and a top block disposed on the solenoid valve, the top block corresponds to the detection kit clamping port, the solenoid valve is configured to push the top block into the mounting groove through the detection kit clamping port when energized, causing the top block to fix or loosen the nucleic acid detection kit.

12. The nucleic acid detection device of claim 8, wherein the sample heating area comprises a holding tank and a heating block disposed at a bottom of the holding tank.

13. The nucleic acid detection device of claim 8, further comprising a first sensor disposed on the detection kit installation area and a second sensor disposed on the sample heating area, wherein the first sensor is configured to sense whether the nucleic acid detection kit is inserted into the detection kit installation area, and the second sensor is configured to sense whether the detection solution is added into the sample heating area.

14. The nucleic acid detection device of claim 8, further comprising a display screen and a camera, wherein the display screen is configured to display the image of the nucleic acid detection kit, the camera is configured to record identification information of the detection solution.

15. The nucleic acid detection device of claim 8, wherein the nucleic acid detection kit comprises:
a kit body;
a sampling port disposed on the kit body;
a detection chip disposed in the kit body;
an electrophoresis box disposed in the kit body;
a detecting window; and
a connector disposed in the kit body,
wherein the detection chip is connected to the sampling area through the sampling port, the detection chip is connected to the electrophoresis box, the detecting window is disposed on a surface of the kit body close to the image collection unit, the detecting window corresponds to the electrophoresis box, the connector is electrically connected to the detection chip and the electrophoresis box.

16. The nucleic acid detection device of claim 8, further comprising a reagent package disposed to store a detection reagent.

17. The nucleic acid detection device of claim 8, wherein the liquid transfer structure comprises:
a lower shell;
an upper shell;
a liquid extraction assembly; and
a pressing key,
wherein the upper shell is movably connected to the lower shell, the liquid extraction assembly is disposed in the lower shell and the upper shell, an end of the liquid extraction assembly extends out of the lower shell, and the pressing key is disposed on the upper shell.

18. The nucleic acid detection device of claim 8, wherein an identification code is set on the collection cup.

19. The nucleic acid detection device of claim 18, wherein the identification code is a QR code.
